(19) United States
(12) Patent Application Publication
(10) Pub. No.: US 2009/0105556 A1
(43) Pub. Date: Apr. 23, 2009

(54) MEASUREMENT OF PHYSIOLOGICAL SIGNALS

(75) Inventors: John Robert Fricke, Lexington, MA (US); Matthew Corbin Wiggins, Concord, MA (US)

Correspondence Address: OCCHIUTI ROHLICEK & TSAO, LLP, 10 FAWCETT STREET, CAMBRIDGE, MA, 02138, US

(73) Assignee: Tiax LLC, Cambridge, MA (US)

(21) Appl. No.: 12/240,651

(22) Filed: Sep. 29, 2008

Related U.S. Application Data
(60) Provisional application No. 60/995,723, filed on Sep. 28, 2007.

(51) Int. Cl. A61B 5/00; A61B 5/1455

(52) U.S. Cl. 600/301; 600/310

(57) ABSTRACT

A system includes an optical sensor and a signal processing module. The optical sensor is configured to be positioned on an area of skin of a patient. The optical sensor includes a light source for illuminating a capillary bed in the area of skin and a photodetector. The photodetector is configured to receive an optical signal from the capillary bed resulting from the illumination and to convert the optical signal into an electrical signal, the optical signal characterizing a fluctuation in a level of blood in the capillary bed. The signal processing module is configured to process the electric signal using a nonstationary frequency estimation method to obtain a processed signal related to at least one of a heart rate and a respiration rate of the patient. Another aspect relates to obtaining a quantity related to the blood pressure of the patient in addition to or instead of obtaining a processed signal related to at least one of the heart rate and the respiration rate of the patient.

### CROSS-REFERENCE TO RELATED APPLICATIONS

This application claims priority to U.S. provisional application No. 60/995,723, filed Sep. 28, 2007, entitled “Method and Devices for Measurement of Multi-modal Physiological Signals,” which is incorporated herein by reference.

### STATEMENT REGARDING FEDERALLY SPONSORED RESEARCH

The subject matter described in this application was partially funded by the Government of the United States under Contract No. W91ZLK-04-P-0239 awarded by the U.S. Department of the Army. The government has certain rights in the invention.

### FIELD OF THE INVENTION

The invention relates to measurement of physiological signals.

### BACKGROUND

Physiological signals are important for monitoring a subject's physical and cognitive state. Often, heart rate parameters are measured directly via electrocardiogram (ECG) measurements of a heart beat. Respiration rate data can be obtained from a respiration chest strap. Physiological signals can also be extracted from infrared (IR) photoplethysmographs (PPG). The signals of interest include heart rate, respiration rate, continuous blood pressure, and intrathoracic pressure. With respect to blood pressure, there is technology related to collecting data at two locations on the body and using pulse transit time and other parameters as the basis of the pressure estimate.

### SUMMARY

In a general aspect, a system includes an optical sensor and a signal processing module. The optical sensor is configured to be positioned on an area of skin of a patient. The optical sensor includes a light source for illuminating a capillary bed in the area of skin and a photodetector. The photodetector is configured to receive an optical signal from the capillary bed resulting from the illumination and to convert the optical signal into an electrical signal, the optical signal characterizing a fluctuation in a level of blood in the capillary bed. The signal processing module is configured to process the electric signal using a nonstationary frequency estimation method to obtain a processed signal related to at least one of a heart rate and a respiration rate of the patient.

Embodiments may include one or more of the following. The system includes an output for providing information determined from the processed signal. The nonstationary frequency estimation method includes a Hilbert transform method or an instantaneous frequency estimation method. The processed signal includes at least one of instantaneous heart rate, inter-beat interval, heart rate variability, high-low heart rate ratios, respiration rate, inter-breath interval, and respiration rate variability. The fluctuation in the level of blood in the capillary bed relates to a change in at least one of volume and pressure of the thoracic cavity or to a change in at least one of volume and pressure of an organ in the thoracic cavity.

The system includes an auxiliary sensor configured to detect an ambient signal. The auxiliary sensor includes at least one of accelerometer, a pressure sensor, an optical detector, a temperature sensor, and a piezoelectric device. The signal processing module is configured to remove an effect of the ambient signal from the electrical signal. The optical signal is a reflectance or a transmittance of the capillary bed.

In another general aspect, a method includes illuminating a capillary bed in an area of skin of a patient, receiving an optical signal from the capillary bed resulting from the illumination, converting the optical signal into an electrical signal, and processing the electrical signal using a nonstationary frequency estimation method to obtain a processed signal related to at least one of a heart rate and a respiration rate of the patient. The optical signal characterizes a fluctuation in a level of blood in the capillary bed.

Embodiments may include one or more of the following. The method includes outputting information determined from the processed signal. Processing the electrical signal using the nonstationary frequency estimation method includes performing a Hilbert transform or processing the electrical signal using an instantaneous frequency estimation method. Processing the electrical signal using the instantaneous frequency method includes band pass filtering the electrical signal, determining an instantaneous frequency of the electrical signal, and using the instantaneous frequency to obtain the processed signal.

The method further includes processing the electrical signal using a model to obtain a blood pressure signal related to a blood pressure of the patient. The optical signal characterizes a capillary refill time in the capillary bed. Processing the electrical signal includes processing the electrical signal in real time.

In another aspect, a method for monitoring blood pressure includes illuminating a capillary bed in an area of skin of a patient, receiving an optical signal from the capillary bed resulting from the illumination, converting the optical signal into an electrical signal, and processing the electrical signal using a model characterizing a relationship of the fluctuation in the level of blood and the blood pressure of the patient to obtain a quantity related to the blood pressure of the patient. The optical signal characterizes a fluctuation in a level of blood in the capillary bed of the patient.

Embodiments may include one or more of the following. The method includes outputting information determined based on the quantity related to the blood pressure of the patient. The optical signal characterizes a capillary refill time. The method further includes engaging a device to restrict circulation in the capillary bed of the patient and disengaging the device prior to receiving the optical signal from the capillary bed. The disengaging of the device occurs gradually. The device is an active clamping device.

The quantity related to the blood pressure of the patient is a quantity related to the continuous blood pressure of the patient. Applying the model includes applying a model including circuit elements or properties of the capillary bed. The method further includes calibrating the model on the basis of a blood pressure of the patient determined by using a blood pressure cuff.

Embodiments may include one or more of the following advantages.

A system or method as described above can be used for both military and civilian applications. Combat casualty care requires close monitoring of vital signs from the moment that a medic first attends to a wounded soldier in the battlefield and thence through the many transfer stages to the point of full hospital care, generally removed from the combat scene. Similar needs are evident in the civilian community where critical care is administered by first responders at the scene of accidents, by emergency room staff, and by intensive care unit staff. It is often desirable to obtain maximum information using as little equipment as possible. The system and method described herein support this need. They reduce the burden of equipment logistics, the burden of extra wires and sensors on and around the patient, and the complexity and cost of using multiple devices.

For both military and civilian applications, a disposable, wearable device in keeping with the system and method described herein can be adapted to stay with a patient and to report vital signs throughout the care and transport processes. Further, the system can be configured to provide medical personnel with real-time visibility of vital signs as well as recording of this information for playback by attending medical staff at a later time. The disposability of the device allows it to be fabricated with low cost parts and eliminates the need for sanitization and asset tracking logistics in large scale clinical or military uses.

Such system and methods additionally support applications in fitness monitoring, where their ease of use and robustness make them a compelling alternative to chest strap monitors for the monitoring of cardiac and respiratory parameters during exercise. An ear-worn device can also integrate a speaker unit for mobile electronic devices such as mobile phones or music players.

An advantage of applying a nonstationary frequency estimation method (e.g., analysis involving monitoring the frequency changes of the signal over time, such as monitoring changes in the instantaneous principal frequency over time) is that it is possible to avoid a tradeoff inherent in many stationary estimation methods between frequency resolution and duration of data signals being analyzed. For example, if the signal is assumed to be stationary within each of a series of data windows, the frequency resolution is generally inversely proportional to the duration of the window. As the window duration increases, the assumption of a stationary signal is increasingly violated and/or nonstationary events (e.g., transients) are more difficult to detect. At least some nonstationary frequency analysis methods, which may be based, without limitation, on a Hilbert transform approach, tracking of a nonstationary model, nonstationary principal frequency analysis, or other time-frequency methods, mitigate the effects of such a time-frequency tradeoff. Furthermore, use of such nonstationary techniques, as opposed to use of time domain peak picking and/or threshold based techniques, can provide robustness of algorithm against artifacts, and provide sensitivity to periodicity without being burdened by a window that can reduce the time resolution.

Other features and advantages are apparent from the following description and from the appended claims.

### BRIEF DESCRIPTION OF DRAWINGS

FIG. 1 is a schematic diagram of a photoplethysmograph (PPG) sensor system.

FIG. 2 is a graph of a PPG detector signal taken over a 25 second period by an earlobe PPG device.

FIG. 3 is a flow diagram of signal processing of a detector signal from a PPG device to obtain heart rate and respiration rate parameters.

FIG. 4 is a graph of a result of band-pass filtering the data shown in FIG. 2 between 0.5 Hz and 5.5 Hz to extract a cardiac signal.

FIG. 5 is a graph of a result of band-pass filtering the data shown in FIG. 2 between 0.17 Hz and 0.5 Hz to extract a respiration signal.

FIG. 6 is a graph of an inter-beat interval obtained by applying an instantaneous frequency method to the cardiac signal shown in FIG. 4.

FIG. 7 is a graph of a spectral analysis of the inter-beat interval data shown in FIG. 6.

FIG. 8 is a graph of the respiration rate obtained by applying an instantaneous frequency method to the respiration signal shown in FIG. 5.

FIG. 9 is a diagram of PPG measurements related to physiological states used to determine intrathoracic pressure.

FIG. 10 is a graph of the output of a matched filtering process using the PPG detector signal shown in FIG. 2 and a pulse pilot signal.

FIG. 11 is a block diagram of a least mean squares (LMS) adaptive filter.

FIG. 12 is a schematic diagram of an active clamping mechanism used to stimulate capillary refill.

FIG. 13 is a diagram of a system model relating a PPG signal to blood pressure.

FIG. 14 is a graph of trends in various physiological parameters before and during a stress event.

FIG. 15 is a block diagram of a portable electronics unit.

FIG. 16 is a flow diagram of methods to estimate a heart rate and a respiration rate.

FIG. 17 is a flow diagram of a processing delay in the estimation of a heart rate.

FIG. 18 is a flow diagram of a processing delay in a first method for the estimation of a respiration rate.

FIG. 19 is a flow diagram of a processing delay in a second method for the estimation of a respiration rate.

FIG. 20 is a flow diagram of a processing delay in a third method for the estimation of a respiration rate.

### DETAILED DESCRIPTION

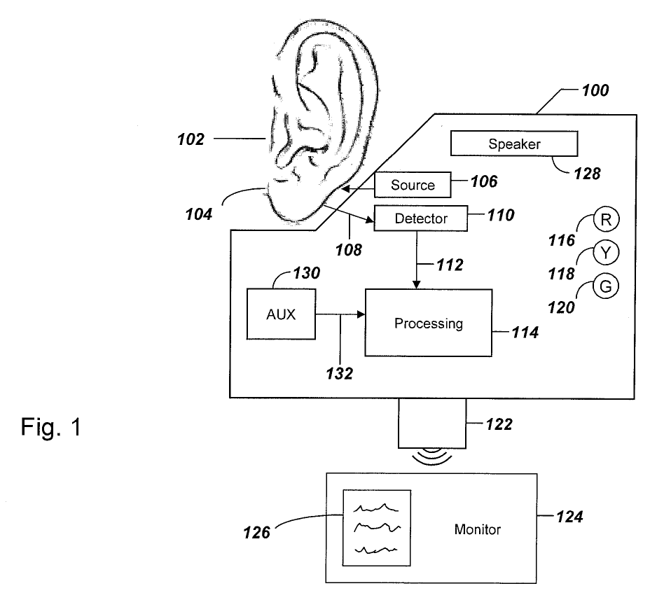

Referring to FIG. 1, examples of an infrared photoplethysmograph (PPG) device 100 are used to obtain physiological signals related to one or more of heart rate, respiration rate, blood pressure, and intrathoracic pressure. Such signals may be relevant for monitoring a person's state, including one or more of the person's physical state, long-term health, psychological state, and/or cognitive state. More generally, the physiological signals may provide information about the activity of the person's sympathetic and parasympathetic nervous system. The PPG device 100 illustrated in FIG. 1 is attached to an earlobe 102 of a person, for example, using a clamping or adhesive approach. However, in other embodiments, PPG device 100 is used on other areas of the skin of a person, including but not limited to a portion of a forehead, a neck, an arm, a forearm, a finger, a leg, a back, an abdomen, or a stomach. In general, a requirement for the positioning of PPG device 100 is that the PPG sensor be located such that it can obtain a measurement via the skin that is related to blood flow or pressure, for example to measure a level of blood in a capillary bed 104, for example, a blood volume, a rate of blood flow, or a rate of change of blood volume. Note also that the approach is not limited to use of a single PPG device on an individual. In some embodiments, multiple PPG devices are used, for example, on the torso and/or at different extremities, and signals obtained at the different PPG devices are processed independently or in combination to determine underlying characteristics of the individual's state.

In some embodiments, such as that shown in FIG. 1, an infrared light source 106 illuminates the earlobe 102. The blood level in capillary bed 104 affects the amount of light 108 that is backscattered or reflected by earlobe 102. Light 108 backscattered by earlobe 102 is received by an optical transducer such as a photodetector 110 and converted into a detector signal 112. Since the blood flow in capillary bed 104 is controlled by the heart beat of the person and thus the blood level in the capillary bed varies with time, the backscattered light 108 and hence the detector signal 112 are also time-varying. In another embodiment, the PPG sensor operates in transmission mode and the light transmitted through the capillary bed is received by the photodetector.

The detector signal 112 is sent to a signal processing unit 114 which processes the detector signal, which contains information about the person's pulse, to extract desired physiological data, in various embodiments including one or more of instantaneous heart rate, inter-beat interval, heart rate variability, high-low heart rate ratio, respiration rate, inter-breath interval, respiration rate variability, blood pressure, and intrathoracic pressure. A single PPG device 100, referred to below as an Integrated Multi-Modal Physiological Sensor (IMMPS), is capable of producing multiple (or all) of such types of physiological data.

In some embodiments, the PPG device 100 provides real-time visibility of physiological parameters and vital signs, which can be transmitted to other equipment for real-time processing or for playback or off-line processing at a later time. In some embodiments, the PPG device includes user output devices, such as a set of light emitting diodes (LEDs) (e.g., a red LED 116, a yellow LED 118, and a green LED 120) or an audio device for producing alert sounds, which provide on-device status on PPG device 100. As an example for use of such output devices, when a selected physiological parameter is in a normal range, green LED 120 is turned on; when the physiological parameter is in a slightly abnormal range, yellow LED 118 is turned on; when the physiological parameter is in a dangerous range, red LED 116 is turned on. In some embodiments, the audio output device is used to provide other audio output, such as the output for an electronic device such as a mobile phone or a music player. In some embodiments, a wireless link 122 to an external monitoring system 124, such as a bedside system or a wearable system, provides sensor data to the external system enabling a numeric readout 126 of various physiological parameters. In some embodiments, the PPG device, or at least some wearable portion of the device, is disposable. In such disposable embodiments, the bedside system can be designed to be sterilized and reused; in another embodiment, the bedside system itself is also disposable. In some embodiments, the bedside system includes or communicates with a centralized monitoring system that monitors PPG devices of multiple patients.

In some embodiments, the photodetector based detector signal is augmented with other signals, for example, accelerometer or pressure sensor signals. For example, auxiliary sensors 130 are connected to signal processing unit 114 via a wired connection 132. In other embodiments, auxiliary sensors 130 are connected to signal processing unit 114 via a wireless connection. Auxiliary sensors 130, such as temperature sensors, accelerometers, pressure transducers, optical detectors, or piezoelectric films or matrices can provide auxiliary signals 132 related to ambient sources of noise to signal processing unit 114. Signal processing unit 114 incorporates auxiliary signals 132 into the signal processing, for example, to increase the signal-to-noise ratio of the desired physiological data.

### Heart and Respiration Rate Signals

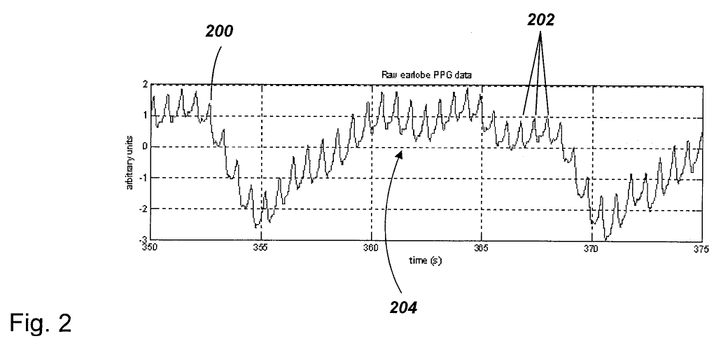

Referring to FIG. 2, in an embodiment of the PPG device that uses a photodetector signal obtained from at an earlobe location, a detector signal 200 obtained from the PPG device 100 has a high-frequency pulse signal 202 whose local peaks in the time domain have a one-to-one correspondence with cardiac beats. Significant low-frequency amplitude variability in the detector signal is due in large part a respiration signal 204, which modulates the baseline of the pulse signal.

The time varying heart and respiration components in detector signal 200 can be modeled as

s(t)=A_{H}(t)cos [ω_{H}(t)t+φH(T)]+A_{R}(t)cos [ω_{R}(t+φ_{R}(T)]+N(t), (1)

where A_{H}(t) is the amplitude modulation of pulse signal 202, ω_{H}(t) is the frequency modulation of pulse signal 202, φ_{H}(t) is the phase modulation of pulse signal 202, A_{R}(t) is the amplitude modulation of respiration signal 204, ω_{R}(t) is the frequency modulation of respiration signal 204, φ_{R}(t) is the phase modulation of respiration signal 204, and N(t) is the time varying noise, which includes baseline drift and broadband noise in the overall signal band.

Given the measured detector signal 200 (s(t)), signal processing is performed to estimate the slowly varying components of the heart rate ω_{H}(t) and the respiration rate ω_{R}(t). It is known that ω_{H}(t)≈ω_{H0}≈1 beat per second 1 Hz for heart rate and ω_{R}(t)≈ω_{T0}≈12 breaths per minute≈0.2 Hz for respiration rate.

Amplitude, phase, and frequency modulation cause spectral spread that broadens the pure tones implied by these frequencies. Amplitude and phase modulation and rapid fluctuations of the frequency modulation are confounding components of detector signal 200. The slowly varying components of ω(t) are the desired components for obtaining heart and respiration rate information.

For both heart and respiration rate, ω(t), is composed of three parts: constant frequency ωₒ, which is the nominal heart or respiration rate; a zero-mean, slowly varying frequency component Ωₛ having a time scale of minutes; and a zero-mean, rapidly varying frequency component Ω_{f} having a time scale of seconds. In this case, the composite heart rate or respiration rate is written as

ω(t)=ωₒ+Ωₛ(t)+Ω_{f}(t). (2)

The signal of interest is the combination of the constant and the slowly varying component (for both heart rate and respiration rate) and is written as

\hat{ω}(t)=ωₒ+Ωₛ(t). (3)

The phase modulation noted is Eq. (1), φ(t), is assumed to be small, since large phase modulation can be represented as frequency modulation and this is already captured in Ωₛ or Ω_{f}.

Applying Eq. (3) to Eq. (1), a new equation for the measured raw signal 200 is obtained:

s(t)=A_{H}(t)cos [\hat{ω}_{H}(t)t+φ_{εH}(t)]+A_{R}(t)cos [\hat{ω}_{R}(t)t+φ_{εR}(t)]+N(t), (4)

where for both heart and respiration rate, small components φ_{ε}(t) are defined as

φ_{ε}(t)=∫_{-∞}^{t}Ω_{f}(τ)dτ+φ(t) (5)

and by construction |φ_{ε}(t)|<<1 and φ_{ε}(t) is zero mean.

Using the Law of Cosines and the fact that |φ_{ε}(t)|<<1, the following equation for raw signal 200 is obtained:

s(t)=A_{H}(t){ cos [\hat{ω}_{H}(t)t]−φ_{εH}(t)sin [\hat{ω}_{H}(t)t]}+A_{R}(t){ cos [\hat{ω}_{R}(t)t]−φ_{εR}(t)sin [\hat{ω}_{R}(t)t]}+N(t). (6)

The formulation in Eq. (6) of PPG detector signal 200 suggests a number of methods to estimate the desired slowly varying heart and respiration rate signals \hat{ω}_{H}(t) and \hat{ω}_{R}(t), respectively. Such estimation techniques can include instantaneous frequency determination via analytic signals, moving averages, band pass filtering, synchronous detection, correlation detection, narrowband processes (e.g., demodulation), matched filtering, wavelet filtering, short-time frequency analysis (e.g., short-time fast Fourier transform, Wigner-Ville transform), state estimation (e.g., Kalman filtering, unscented filtering), Doppler processing, or a combination of the above methods. A number of these techniques can be implemented to account for the nonstationary nature of the detector signal, which relates to the time variation of the frequency modulation signals, ω(t).

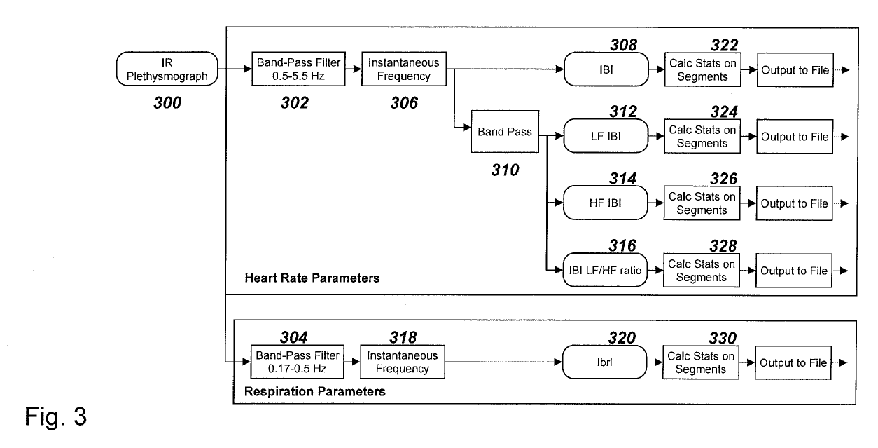

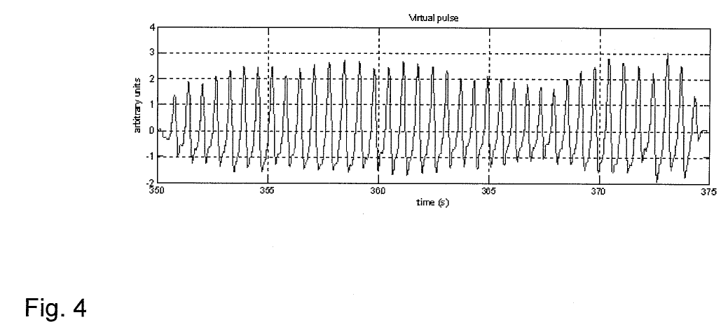

Referring to FIG. 3, one example of a procedure for obtaining heart rate parameters and/or respiration rate parameters involves receiving the detector signal 200, such as that shown in FIG. 2, from a PPG photodetector (IR Plethysmograph 300). To emphasize the pulse signal in order to extract cardiac parameters from the detector signal, the respiratory modulation is removed by band-pass filtering (302) the detector signal between approximately 0.5 Hz and approximately 5.5 Hz. Band-pass filtering of the detector signal shown in FIG. 2 results in a cardiac signal shown in FIG. 4, which has significantly less amplitude variability due to respiratory contamination. The band-pass filtered signal pulse signal can be expressed as

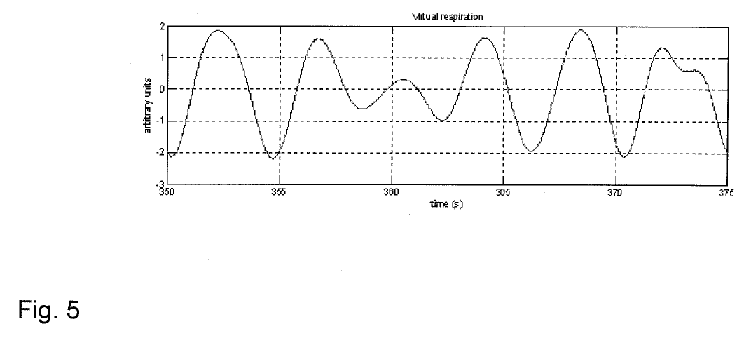

\tilde{s}(t)=A_{H}(t){ cos [\hat{ω}_{H}(t)t]−φ_{εH}(t)sin [\hat{ω}_{H}(t)t]}+Ñ(t), (7)

where Ñ(t) is the content of the noise, N(t), within the heart rate bandwidth. Likewise, referring again to FIG. 3, to extract parameters related to respiration from a PPG detector signal, the pulse signal is removed by band-pass filtering (304) the detector signal between approximately 0.17 Hz (equivalent to 10 breaths per minute) and 0.5 Hz (30 breaths per minute). Band-pass filtering the detector signal of FIG. 2 produces a respiration signal shown in FIG. 5, which retains primarily low frequency respiration components of the original signal.

Focusing now on the cardiac signal, one approach to detecting heart beats in the cardiac signal is by threshold-based peak picking, which can be used to determine the time of specific events such as heart beats. In some implementations, peak picking can be sensitive to the threshold selected. For example, if the threshold is set too low, then false beats can be counted, and the inter-beat interval (IBI) is determined to be shorter than it really is. If the threshold is set too high, then true beat peaks may be missed. For general processing it is convenient to select a single threshold, but if the data are sufficiently variable then selecting a single threshold is not possible.

Other approaches to processing the cardiac signal use nonstationary frequency estimation methods. For instance, instantaneous frequency computation component 306, 318 are used to determine the time-varying principal frequencies present in bandpass filtered signals. Approaches to instantaneous frequency computation include Hilbert Transform methods, which are particularly effective because the band-pass filtered cardiac signal shown in FIG. 4 has a strong sinusoidal content and because systematic changes in the IBI over long periods of time are often of interest. Model-based approaches, such as Kalman filtering approaches described later in this document can also be used, with state variables (or alternatively time-varying system parameters) that are estimated corresponding to the instantaneous frequencies of interest. Yet other approaches to tracking instantaneous frequency can be based on adaptive modeling of the underlying quasi-periodic heart signal.

In some embodiments, an approach to determining the instantaneous frequency relies on analytic signals, which are signals that have no negative frequency components. Based on the properties of the Fourier transform, a signal with no negative frequencies is a complex signal in the time domain. Given a real signal, xᵣ(t), the corresponding analytic signal, xₐ(t)=xᵣ(t)+jxᵢ(t), has the same positive frequency spectrum as xᵢ(t) but has zero negative frequency values. Thus, the imaginary signal xᵢ(t) must be determined. The utility of computing the analytic signal becomes apparent when it is written as follows:

$$\begin{matrix}{x}_{a} ( t ) = {x}_{r} ( t ) + j {x}_{i} ( t ) \\ = A ( t ) {e}^{jφ ( t )} ,\end{matrix} \qquad ( 8 )$$

where A(t) is the time varying magnitude of the complex signal, i.e., the envelope, and φ(t) is the time varying instantaneous phase of the complex signal. By construction, the magnitude and instantaneous phase can be written as:

A(t)=\sqrt{xᵣ²(t)+xᵢ²(t)} and

φ(t)=tan⁻¹(xᵢ(t)/xᵣ(t)) . (9)

The instantaneous phase may be used to compute the instantaneous frequency by recognizing that

$$ω ( t ) = \frac{d φ ( t )}{d t} . \qquad ( 10 )$$

The signal magnitude, A(t), and the instantaneous frequency, ω(t), are often useful quantities.

Because of the properties of the Fourier transform, the spectrum of a real signal has conjugate symmetry; that is, the negative spectral values are the complex conjugate of the positive values. Given that the definition of the analytic signal is that there are no negative frequencies, the imaginary time series, xᵢ(t) must have the same spectrum as the real time series, xᵣ(t), but with a +π/2 phase shift for negative frequencies and a −π/2 phase shift for positive frequencies. When added to the transform of the real component, the desired result is obtained. These properties of the imaginary component, xᵢ(t), are a description of the Hilbert transform of the real component, xᵣ(t). Specifically, the Hilbert transform is written in the frequency domain as:

$$\begin{matrix}H ( ω ) = - j sgn ( ω ) , \\ where & ( 11 ) \\ sgn ( ω ) = \{ \begin{matrix}1 , & for ω > 0 \\ 0 , & for ω = 0 \\ - 1 , & for ω < 0.\end{matrix} & ( 12 )\end{matrix}$$

An advantage of the instantaneous frequency method is that a threshold does not have to be set. In the peak picking method, the selection of the threshold is often somewhat arbitrary and may not be dictated by underlying physiological or physical processes. In contrast, the tuning parameter used for the instantaneous frequency method is the selection of the frequency band for filtering the data. Since it is known that heart rate lies within physiological limits, the filter selections are prescribed rather than arbitrary.

Specifically considering the case of the heart rate signal, the expression \tilde{s}(t), also called \tilde{s}ᵣ(t), is the real part, and an imaginary part \tilde{s}ᵢ(t) is formed via a Hilbert transform as described above. A −π/2 phase shift is introduced for positive frequencies and a +π/2 phase shift is introduced for negative frequencies. Explicitly,

\tilde{s}ᵣ(t)=A_{H}(t){ cos [\hat{ω}_{H}(t)t]−φ_{εH}(t)sin [\hat{ω}_{H}(t)t]}+Ñᵣ(t)

\tilde{s}ᵢ(t)=A_{H}(t){ sin [\hat{ω}_{H}(t)t]+φ_{ε}H(t)cos [\hat{ω}_{H}(t)t]}+Ñᵢ(t). (13)

The instantaneous phase of this complex signal is

$${\tilde{φ}}_{H} ( t ) ≅ {tan}^{- 1} [ \frac{{\tilde{s}}_{i} ( t )}{{\tilde{s}}_{r} ( t )} ] \\ = {tan}^{- 1} [ \frac{sin [ {\hat{ω}}_{H} ( t ) t ] + {φ}_{ɛ H} ( t ) cos [ {\hat{ω}}_{H} ( t ) t ] + \frac{{\tilde{N}}_{i} ( t )}{{A}_{H} ( t )}}{cos [ {\hat{ω}}_{H} ( t ) t ] - {φ}_{ɛ H} ( t ) sin [ {\hat{ω}}_{H} ( t ) t ] + \frac{{\tilde{N}}_{r} ( t )}{{A}_{H} ( t )}} ] . \qquad ( 14 )$$

By construction, |φ_{εH}(t)|<<1, and with good quality measurements Ñᵢ(t)/A_{H}(t) and Ñᵣ(t)/A_{H}(t) are small. In this case,

$$\begin{matrix}{\tilde{φ}}_{H} ( t ) ≅ {tan}^{- 1} \{ tan [ {\hat{ω}}_{H} ( t ) t ] \} \\ = {\hat{ω}}_{H} ( t ) t ,\end{matrix} \qquad ( 15 )$$

which allows the instantaneous frequency to be determined as

$${\hat{ω}}_{H} ( t ) = \frac{d {\tilde{φ}}_{H} ( t )}{d t} \qquad ( 16 )$$

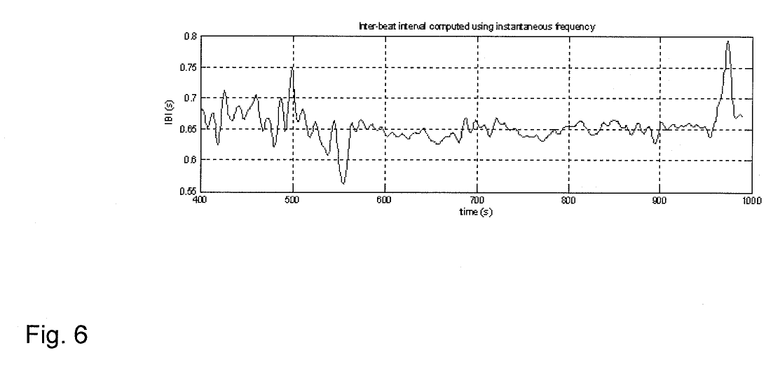

Referring to FIG. 6, the IBI (seconds per beat) resulting from applying the instantaneous frequency method to the filtered data of FIG. 4 is shown.

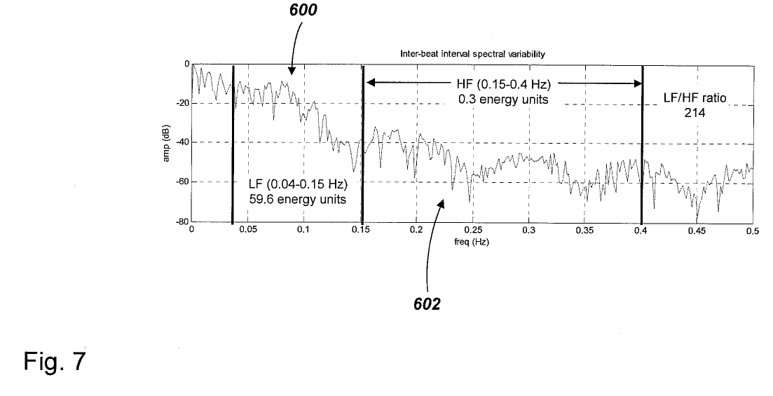

Various spectral bands of the instantaneous heart rate or instantaneous inter-beat interval (IBI) are correlated to physiological state, including low-frequency variability, high-frequency variability, and the power ratio of these two bands. Referring again to FIG. 3, in some embodiments, the IBI computed based on an instantaneous frequency calculation may be used directly as a relevant physiological parameter (308). Alternatively, the IBI is band-pass filtered (310) to select either a low-frequency band (312) or a high-frequency band (314). A ratio of the energy of the low-frequency band to that of the high-frequency is computed (316). The low- and high-frequency components of heart rate variability provide information about the parasympathetic and sympathetic nervous system. Referring to FIG. 7, a spectral decomposition of the derived IBI of FIG. 6 into a low frequency band 600 of approximately 0.04 Hz to approximately 0.15 Hz (6.7 second to 25 second period) and a high frequency band 602 of approximately 0.15 Hz to approximately 0.4 Hz (2.5 second to 6.7 second period) is shown. In order to create a metric for subsequent correlation, the energy in both the low frequency band and the high frequency band is computed. Additionally, the ratio of the energy of the low frequency band to the energy of the high frequency band is determined. For the data shown in FIG. 7, the low frequency energy is 60 mJ, the high frequency energy is 0.28 mJ, and the ratio of low frequency energy to high frequency energy is 214. In the energy computation, it is assumed that the IBI signal of FIG. 6 is a voltage signal with the mean removed and that the power is dissipated across a 1Ω resistor for the 600 second duration of the signal. Expressing the band energy in mJ is a convenience. The ratio of low frequency energy to high frequency energy is independent of the assumptions of the energy computation assumptions and depends only on the linearity of the system.

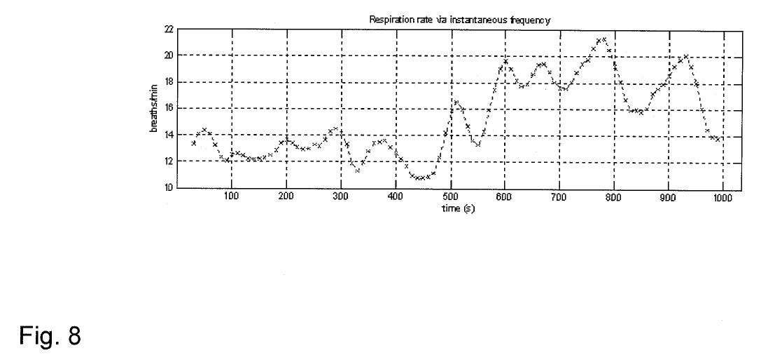

Referring again to FIG. 3, the above instantaneous-frequency based analysis is applied also to respiration parameters. After band-pass filtering the detector signal (304) to obtain the respiration signal, an inter-breath interval (IBrI) is computed using the instantaneous frequency method (318). The IBrI is used directly to produce relevant physiological information (320), such as the respiration rate, as shown in FIG. 8. Specifically, in this example, the marked increase in respiration rate starting around 500 seconds corresponds to the start of a known challenge event in the testing period.

### Variability Statistics

Statistics to quantify heart rate variability and respiration rate variability provide clinical practitioners insight into the physiological state of the individual being monitored. For heart rate, the statistics of the inter-beat interval (IBI) are relevant; for respiration rate, the statistics of the inter-breath interval (IBrI) are relevant. Referring again to FIG. 3, statistics are calculated on any or all of the IBI (322), the low-frequency band of the IBI (324), the high-frequency band of the IBI (326), the ratio of the energy of the low-frequency band to that of the high-frequency band of the IBI (328), and the IBrI (330).

For a given data epoch at time t, the mean of the IBI or IBrI, x(t), is

$$μ ( t ) = \frac{1}{N} {\sum }_{n = 1}^{N} x ( t + n Δ t ) , \qquad ( 17 )$$

where Δt is the sampling interval, N is selected to span the desired epoch, and successive values of t may be selected to result in overlapped epochs, time continuous epochs (end-to-end coverage with no gaps), or epochs separated by time gaps. The standard deviation of the data epoch is computed using the unbiased estimate of the sample variance as

$${σ}^{2} ( t ) = \frac{1}{N - 1} {\sum }_{n = 1}^{N} {[ x ( t + n Δ t ) - μ ( t ) ]}^{2} , \qquad ( 18 )$$

and the standard deviation, σ(t), is the square root of the variance. As with the estimate of the mean, the epochs may overlap, have no gaps, or have gaps according to the choice of N and t. Other statistics including but not limited to skew and kurtosis can also be computed for the IBI and the IBrI.

### Deterministic and Stochastic Processing

Although the PPG signal is generally robust to motion artifacts, in some circumstances external vibration contaminates the signal, such as the high vibration levels during transport of a patient. A particularly challenging vibration environment occurs during transport via helicopter; vibrations in this situation are characterized by a broadband, low-level noise floor combined with strong tonal components driven by the rotating machinery of the aircraft. Within the cargo compartment of a helicopter, the dominant vibration sources are the main rotor fundamental frequency, the blade passage frequency, and harmonics of these. For instance, for a Blackhawk UH-60Q MEDEVAC helicopter, the rotor fundamental frequency is 4.3 Hz and, with four blades, the blade passage frequency is 17.2 Hz.

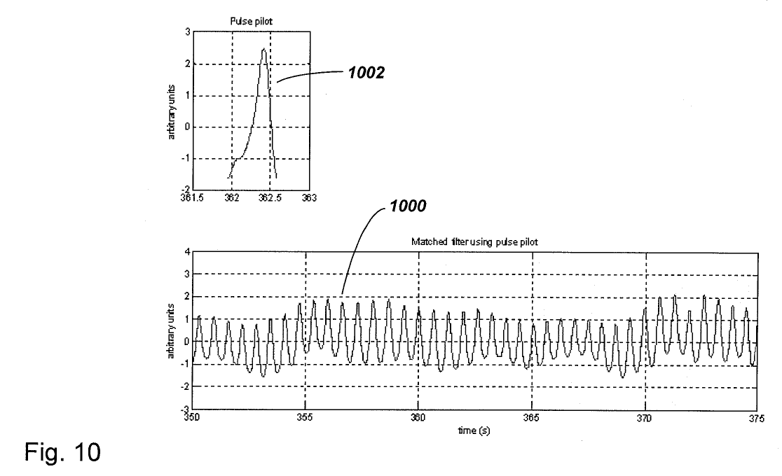

External vibration gives rise to two different effects: generation of noise in the PPG signal and the dislodging of the PPG sensor from the ear. Amelioration of vibrational noise effects is handled through deterministic and stochastic processing. For instance, the band-pass filtering described above is one form of deterministic processing. Another deterministic approach is matched filtering with a pilot signal representative of the individual PPG signal wavelets. Matched filtering, a correlation technique, is expressed in the following equation:

$$v ( t ) = {\int }_{- ∞}^{∞} s ( τ ) x ( t + τ ) d τ , \qquad ( 19 )$$

where s(t) is the pilot signal and x(t) is the input PPG signal. The output signal, v(t), has a high value at times corresponding to times in the input signal that have signal character similar to that of the pilot signal. Referring to FIG. 10, a matched filtering output signal 1000 is obtained using the PPG detector signal of FIG. 1 and a pulse pilot signal 1002.

A stochastic processing approach based on Least Mean Square (LMS) adaptive signal processing assumes that the desired signal has been contaminated by another signal, which can be removed adaptively. The contamination signal is assumed to be related to a reference signal that is measured in such a way as not to include any contribution from the desired signal. Additionally, artifacts due to the contamination signal must be linearly related to one or more reference signals that are available contemporaneously with the signal to be filtered. The independent reference signal(s), such as acceleration, pressure, and ambient light, are presented to the LMS signal processing algorithm to remove artifacts in the measured signal and to improve the signal-to-noise ratio (SNR) of the desired PPG signal. This technique is used to reduce vibration-induced effects on the PPG signal when an independent measurement of the vibration signal can be obtained. One example of a reference signal is provided by an accelerometer integrated within the PPG sensor package.

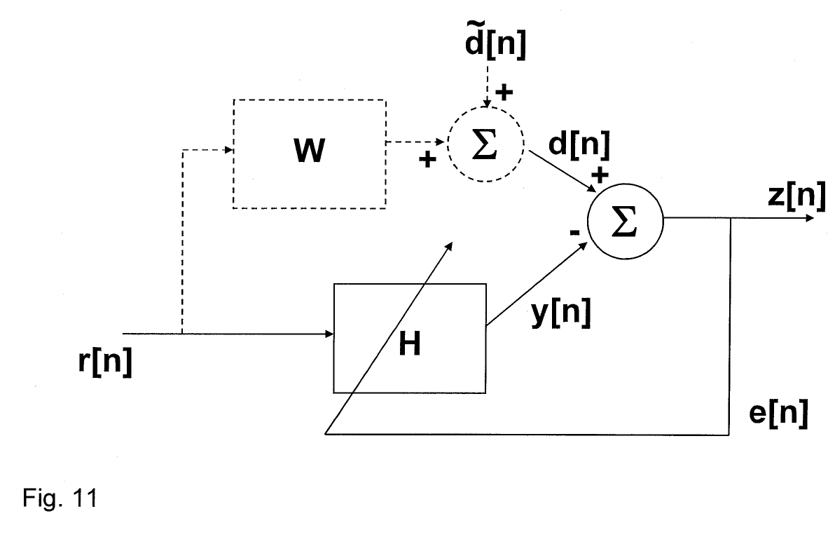

Referring to FIG. 11, the fundamental block diagram for the LMS algorithm depicts the following signals at time step n:

r[n] is one or more reference signals used as the basis for artifact removal

d[n] is the desired signal, \tilde{d}[n], contaminated by noise

y[n] is the best estimate of the contaminating signal given the reference signals

e[n]=z[n] is the cleaned desired signal with contaminating artifacts removed (to the extent possible).

A linear operator W, which may be time-varying, relates the reference signal r[n] to the contaminating signal that is added to the desired signal, \tilde{d}[n], to produce the observed noisy signal d [n]. W is arbitrary and it is not necessary that it be known. The selection of the weights in a second linear operator H is the key to the algorithm. The filter weights of H, i.e., hₙ[i], are computed by minimizing the error between the noisy input signal d[n] and the filtered reference signal y[n]. Specifically, for time step n, the squared error is defined as

eₙ=dₙ−yₙ and eₙ²=(dₙ−yₙ)². (20)

The coefficients hₙ[i] are found by taking the partial derivative of e² with respect to the individual coefficients, providing the gradients. Specifically,

$$\begin{matrix}\frac{\partial }{\partial h [ i ]} {e}^{2} = e \cdot 2 \frac{\partial e}{\partial h [ i ]} \\ = e \cdot 2 \frac{\partial }{\partial h [ i ]} ( d - y ) \\ = e \cdot 2 \frac{\partial }{\partial h [ i ]} ( d - {\sum }_{i = 0}^{N - 1} ( h [ i ] r [ n - i ] ) ) \\ = - 2 er [ n - i ]\end{matrix} \qquad ( 21 )$$

The final result of Eq. (21) provides the gradient for the coefficients that minimize the mean square error, e. The order of the filter is determined by the number of prior input values used at each time step (which determines the number of coefficients in hₙ. Thus, given any particular starting point for the coefficients hₙ[i], the gradients are used to move in the direction towards minimizing error.

The remaining unknown is how to start the search. A convenient starting point is to assume that all the hₙ[i] are identically zero. The data and algorithm will then guide toward a solution for incrementally reducing and maintaining the error at a minimum value. For time step n, the filter update equation to determine the coefficients to be used at time step n+1 is written as

hₙ₋₁[i]=hₙ[i]+μeₙr[n−i], (22)

where μ is an update parameter that controls the speed of convergence. To enhance the rate of convergence, the normalized LMS algorithm may be used. This is implemented by normalizing the input values r[n]. The normalization factor is computed as the sum of the squares of the prior input values used for each update step. Specifically, if the order of the filter is N, then the coefficients r on the right hand side of Eq. (22) are normalized by

$$\hat{r} [ j ] = \frac{r [ j ]}{{\sum }_{i = 0}^{N - 1} {r}^{2} [ n - i ]} \qquad ( 23 )$$

where j is in the range n−N+1≦j≦n. These normalized input values are used in place of the r values in Eq. (22).

The use of the LMS filter as described above is applied to a PPG signal that is contaminated by, for instance, some combination of motion, as measured by an accelerometer, pressure fluctuations, as measured by a pressure sensor, and ambient light, as measured by an optical detector. The signal output from each sensor is independent of the hemodynamic signal that is the desired output from the PPG sensor. The PPG signal itself, however, is not independent of these corrupting signals. The LMS algorithm dynamically adapts to the changes in ambient conditions for motion, pressure, and light, and removes these effects from the contaminated PPG signal. In this example, the reference vector r[n] is constructed as the concatenation of three vectors, one of each of the individual reference signals. Specifically, let the reference vector be defined as

r[n]≡[a[n], a[n−1], . . . a[n−M], p[n], p[n−1,] . . . p[n−M], c[n], c[n−1], . . . c[n−M]]^{T}, (24)

where the variables a, p, and c are the current and past M values of acceleration, pressure, and light, respectively. The LMS equation automatically adjusts the weights of hₙ[i] to optimally estimate the influence of a, p, and c on the contaminated signal d. This optimal estimate is then removed at each time step n to produce the improved SNR estimate of the desired signal z.

In other embodiments, more or less than three contaminating signals may be used, according to the situation.

### Blood Pressure

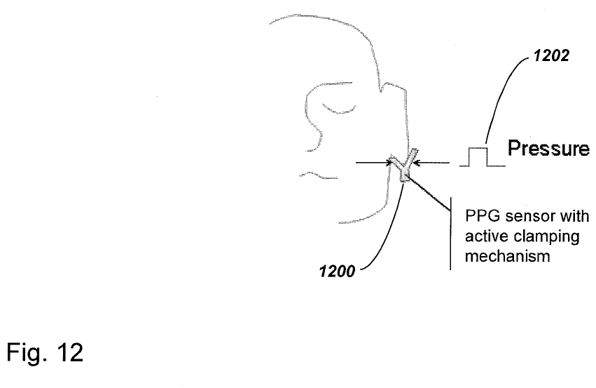

Capillary refill time has a moderate, direct correlation with blood pressure. In some embodiments, this correlation is used to estimate blood pressure from a PPG detector signal. Referring to FIG. 12, an active clamping mechanism restricts circulation in the capillary beds being interrogated by a PPG sensor. For instance, for an earlobe clip 1200, the clamping mechanism can be an adjustable spring, an electric drive, or another variable pressure mechanism. The clamping mechanism is energized to increase the clamping pressure while at the same time the PPG signal is observed. When the pulsatile characteristic of the signal is eliminated, the clamping pressure is released. The PPG signal gives an indirect measurement of the patient's blood pressure. In an embodiment, the clamping mechanism is configured to occlude blood flow and then to slowly release the pressure. A pressure sensor is used to identify the pressure at which the blood flow returns to normal, thus identifying the patient's systolic and diastolic blood pressure for calibration purposes. For instance, a pressure pulse 1202 is applied to the earlobe. The PPG signal is then observed and the capillary refill time (CRT), which is a surrogate for blood pressure, is measured.

In combination with a hemodynamics model of the tissue, such as the Windkessel model, the PPG signal and the associated CRT are converted to a blood pressure measurement. The PPG sensor and processing algorithms described below allow continuous (i.e., non-discrete) blood pressure measurements. By performing a one-time measurement using a cuff-based instrument, additional information may be included in the estimation of blood pressure. For instance, an initial cuff-based blood pressure measurement is used to set calibration parameters for the use of a PPG sensor on a particular patient. In another example, an initial ‘well-being’ indication is entered to calibrate the PPG sensor to factors such as the patient's age or previous blood pressure readings taken at similar stress or activity levels. The active clamping mechanism is not restricted to an earlobe clip; in other embodiments, local pressure concepts are implemented for PPG sensors placed on the forehead, forearm, or other locations on the body. The same PPG sensor may also be used to measure heart rate parameters and respiration rate parameters as described above.

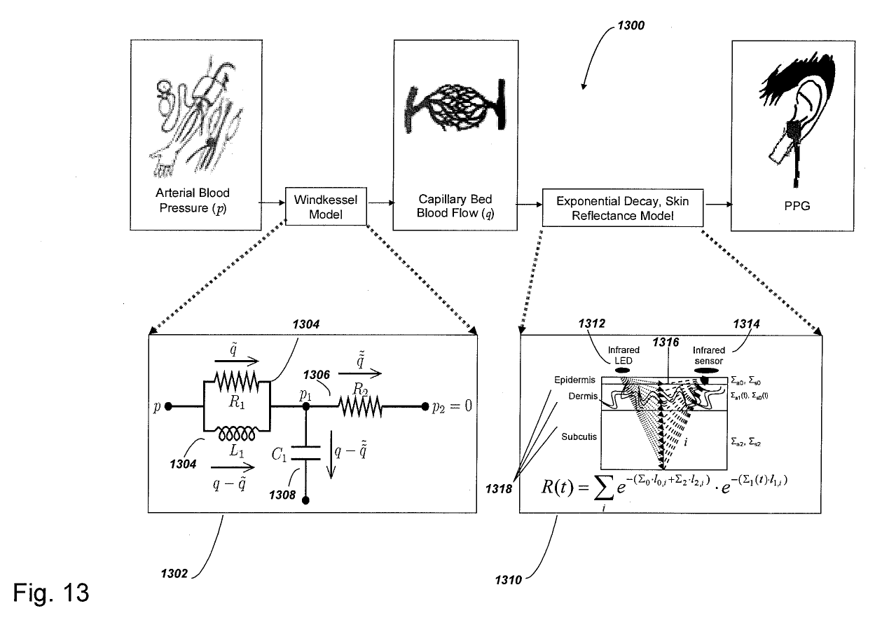

Referring to FIG. 13, a model 1300 that describes the relationship between arterial blood pressure and the reflectance measured by a photo detector at a capillary bed enables the estimation of the continuous arterial blood pressure. In some examples, the model 1300 is designed to be minimalist to enable low-power usage, and in some embodiments contains algorithms for near real-time computation, as described below. Model 1300 is composed of two subsections. A first subsection 1302 is a modified Windkessel model which relates the arterial pressure to the blood flow in the circulatory system through a lumped parameter circuit relationship. The components of this model reflect certain characteristics of blood flow. An inductor 1304 represents the inertia of the blood following contraction of the heart; this inertia carries the blood through the arterial system. Resistors 1304 and 1306 represent resistance due to vessel branching or viscous drag as blood moves through a blood vessel. A capacitor 1308 represents the stretching or compliance of a blood vessel as the compression wave moves down an artery. The “load” portion, represented by a resistor having a resistance R2, can take on several forms, such as a parallel resistor capacitor or other such sub-circuit. The details of this terminal impedance are dependent on the nature of the capillary bed being interrogated by the PPG sensor.

A second subsection 1310 relates the reflectance of light from an infrared light source 1312 back to a photodetector 1314 from blood in a capillary bed 1316. For the embodiment having a PPG sensor that functions in transmission mode, second subsection 1310 instead includes the transmission of light through the capillary bed. This model is based on the path length a photon travels through the different layers 1318 of skin and the light propagation, diffusion, and scattering characteristics of these layers. For instance, factors such as blood pooling and oscillatory blood flow in the capillary bed, the presence and pigmentation of the skin layers, and probabilistic photon paths affect the interaction of the capillary bed with the incident light. Generally, most layers of the skin are assumed to have static characteristics with the absorption characteristics of a single layer changing due to the alteration of blood content. In the embodiment shown in FIG. 13, the absorption characteristic is described as an exponential decay, but other types of path and absorption dependent functions are also possible.

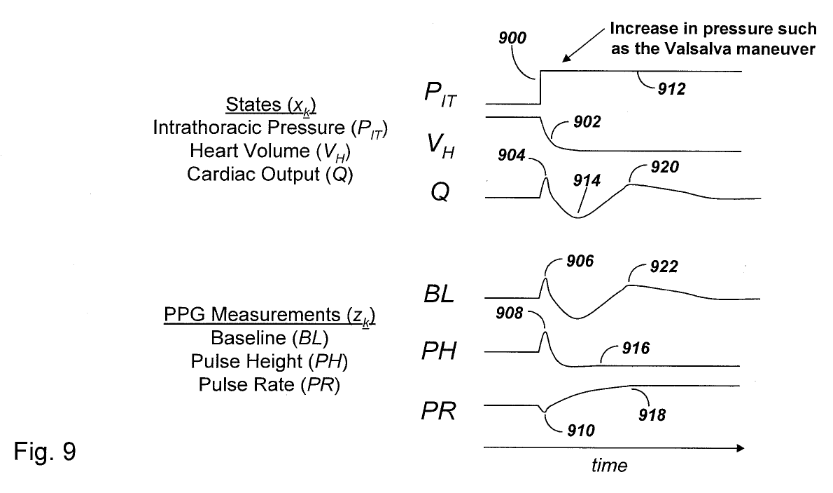

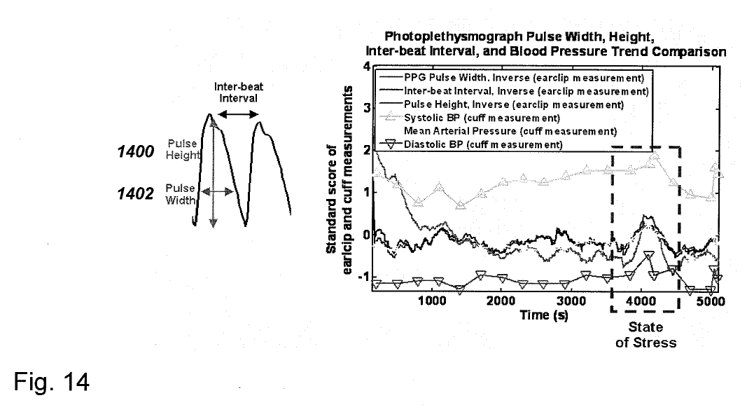

FIG. 14 shows a graph of trends in various physiological parameters before and during a stress event (increased blood pressure) similar to that presented in FIG. 9. A model is based on an empirical relationship between blood pressure versus attributes of a PPG signal, such as pulse height 1400, pulse width 1402, and inter-beat interval 1404. As a subject's blood pressure rises and falls under a state of stress, as seen in the boxed region at about 4000 seconds, the inverse of these attributes follows the trend of the blood pressure. These individual features and their composites show changes in the continuous blood pressure from a previous baseline.

Some or all of these indicators of blood pressure derived from the PPG signal are used as noisy observations of an underlying system state in a Kalman filter to estimate a subject's blood pressure following an initial calibration. This approach involves using the models described above as well as heuristic models describing more general relationships of PPG pulse wave morphology to changes in blood pressure. The system state variables of the Kalman filter include, but are not limited to, arterial blood pressure; capillary bed blood flow; values of the lumped circuit model components such as resistors, capacitors, and inductors as shown in subsection 1302 of FIG. 13; the absorbance and/or thickness of different layers of skin, the inter-beat interval, the pulse height, and the pulse width.

More specifically, in some embodiments a standard Kalman filter is used in which a prediction step and a correction step are used iteratively to obtain estimates of physiological state parameters. To begin, estimates of an initial state and an error covariance are inputted into the model. During a prediction step, a future state \hat{x}ₖ at time step k is predicted from a state \hat{x}ₖ₋₁ at time step k−1 using a system matrix A, a driving matrix B, and a driving noise uₖ:

\hat{x}ₖ⁻=A\hat{x}ₖ₋₁+Bûₖ. (25)

An error covariance Pₖ⁻ is also predicted:

Pₖ⁻=APₖ₋₁A^{T}+Q, (26)

where Q is a noise term and A^{T} is the transverse of A. Following the prediction step, a correction step is performed. A Kalman gain Kₖ is calculated:

Kₖ=Pₖ⁻H^{T}(HPₖ⁻H^{T}+R)⁻¹, (27)

where His an observation matrix and R is the covariance of the observation noise. The estimated state \hat{x}ₖ is then corrected using measurements zₖ obtained from the PPG sensor:

\hat{x}ₖ=\hat{x}ₖ+Kₖ(zₖ−H\hat{x}ₖ⁻) (28)

The error covariance is also corrected using:

Pₖ=(I−KₖH)Pₖ⁻. (29)

(where I is the identity matrix). The results of the correction step are inputted into the prediction step to advance the model by one time step.

The Kalman filter is used to estimate blood pressure and the circuit parameters of the model 1300 shown in FIG. 13 based on measurements obtained from a PPG sensor. Referring to the model 1300, inductor 1304 has an inductance L₁, resistors 1304 and 1306 have resistance R₁ and R₂, respectively, and capacitor 1308 has a capacitance C₁. A frequency domain transfer function H(ω) relates blood pressure P(ω) to blood flow Q(ω):

$$H ( ω ) = \frac{Q ( ω )}{P ( ω )} = {R}_{1} - \frac{{R}_{1}^{2}}{{L}_{1} ( jω + \frac{{R}_{1}}{{L}_{1}} )} + \frac{1}{{C}_{1} ( jω + \frac{1}{{R}_{2} {C}_{1}} )} . \qquad ( 30 )$$

Parameters of second subsection 1310 of model 1300, which deals with skin reflectance, may also be included, allowing for changes in perfusion due for instance to pressure applied by the PPG sensor. In some instances, a pressure sensor is required as an input to the filter to account for these changes. Alternatively, states of the Kalman filter include deviations of the circuit parameters of model 1300 (i.e., ΔR, ΔC, ΔL, etc.) around an experimentally determined physiological mean.

### Interthoracic Pressure Monitoring

Changes in volume and/or pressure due to physical changes or substances (e.g., blood, air, food, or lymph) moving into and out of a thoracic cavity of a person are monitored through changes in physiological parameters derived from a PPG signal, such as pulsatile magnitude, pulse rate, and baseline wander magnitude. The pressure/volume relationship is modeled using the Ideal Gas Law; the volume or pressure of parts of thoracic cavity are kept constant while others are changed. For instance, in the Valsalva and Muller maneuver, the rate of change of volume of the lungs is held constant while the rate of change of volume of thoracic cavity rises and falls, thus reducing and increasing, respectively, the pressure in thoracic cavity. This pressure change affects all the organs in thoracic cavity, including the lungs, heart, and stomach. In particular, the pressure change alters the stroke volume of the heart, resulting in a change in the amount of blood delivered to tissues of the body. In turn, the autonomic nervous system responds by appropriately altering the heart rate to maintain homeostasis. Thus, the baseline or low frequency changes of a PPG signal, the pulsatile signal magnitude, and the pulsatile rate variation can all be used to monitor the intrathoracic pressure.

In another embodiment, the volume of one portion of the system is varied in a known way in order to uncover the response or nature of another component of the system, as in a phase-locked loop. For instance, breathing at a known rate into and out of a bag having a known volume provides known characteristics from which other values can be calculated.

FIG. 9 shows states xₖ, which include interthoracic pressure P_{IT}, heart volume V_{H}, and cardiac output Q; and PPG measurements zₖ, which include a baseline BL, a pulse height PH, and a pulse rate PR. When an increase in thoracic pressure occurs at point 900, for instance due to the Valsalva maneuver, blood is pushed out of the thoracic cavity (which includes the lungs, heart, and blood vessels) and into the rest of the body, causing a rapid drop in heart volume 902, a rapid increase in cardiac output 904. In a PPG signal, this increased blood flow is visible in a increase in a baseline 906, an increased pulse height 908, and a slight drop in pulse rate 910. Following this initial increased cardiac output, the interthoracic pressure remains high 912, preventing the heart from refilling fully with blood to pump to the body. This causes a reduced cardiac output 914. In the PPG signal, the reduced cardiac output is manifest through a decreased pulse height 916 and an increased pulse rate 918. The pulse rate increases to maintain cardiac output. Peaks 920 and 922 in the cardiac output and the baseline, respectively, are a result of the body overcompensating for the earlier insufficient blood flow by increasing the heart rate and stroke volume. Relationships between parameters available through PPG measurements and physiological states, such as those described in FIG. 9, are modeled by a Kalman filter to estimate the intrathoracic pressure.

### Real-Time Processing

In some embodiments, a wearable PPG sensor provides real-time data analysis using causal and efficient computations. Computations are causal due to the fact that data values from the future are unavailable during the computation. For physiological processes in which a delay of a few seconds between a measurement and a computed result is acceptable, the causal feature can be relaxed, but only to the point where an acceptable latency exists in the system. Real-time processing pertains to the fact that the computations are accomplished in less time than the interval between successive samples. If this is not achieved, then the input data accumulates faster than the processed output, resulting in an incrementally increasing latency as time progresses. Ultimately, memory storage limitations prevent further data acquisition.

The principal signal processing algorithms that are performed in real-time include but are not limited to low-, band-, and high-pass filtering; the Hilbert transform; LMS adaptive filtering; Kalman filtering; matched filtering; and sample statistics. Each of these algorithms are performed with various digital signal processing methods with, at worst, finite latency, as shown in Table 1.

TABLE 1

| | Digital | |
|---|---|---|
| Process | algorithm | Comment |
| Spectral filtering | IIR, FIR | no latency for IIR; finite latency for FIR |
| | | to await data |
| Hilbert transform | IIR, FIR | no latency for IIR, finite latency for FIR |
| | | to await data |
| LMS filtering | IIR | computation latency at each time step |
| Kalman filtering | IIR | computation latency at each time step |
| Matched filtering | digital | finite latency to await data |
| | integration | |
| Sample statistics | digital | finite latency to await data |
| | summation | |

The orders of the LMS and Kalman filters have particular bearing on whether the computation latency is greater than the sampling interval. An additional consideration is the processing capability of the microprocessor on which the algorithms are run. As a result, the selection of digital signal processing algorithms is a trade-off between system performance, computational latency, and battery power. Implementation decisions depend greatly on application requirements.

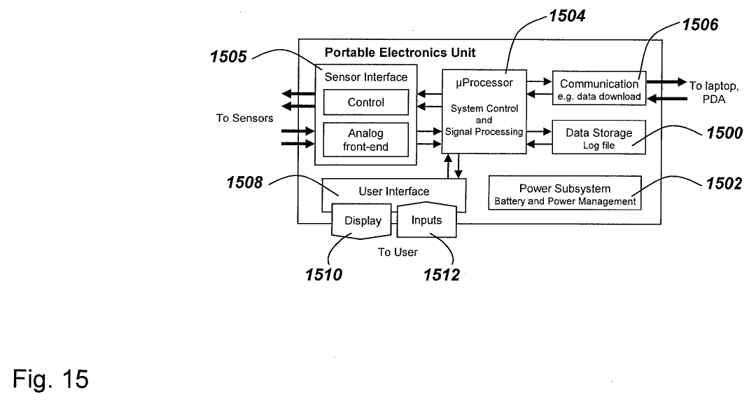

Referring to FIG. 15, some aspects of this engineering tradeoff are appreciated through the block diagram of system elements in a portable electronics unit that receives signals from a PPG device. For real-time computation, a memory 1500 cannot be filled faster than an average data processing rate. A power subsystem 1502 is sized to accommodate system requirements for the duration of the intended application. A microprocessor 1504 is sufficiently powerful to keep up with the sensor data rate received through sensor interface 1505, but not so powerful as to unnecessarily draw down available power stored in power subsystem 1502. Communications with an external host, such as a laptop, are performed with a communications module 1506. These communications are sufficiently fast so that a backlog of processed information is not created, which would overflow the data storage capacity available in memory 1500. A user interface 1508, including a display 1510 and an input module 1512, is designed to be intuitive and informative. The display technology used in display 1510 is selected so as not to unnecessarily draw down the battery power.

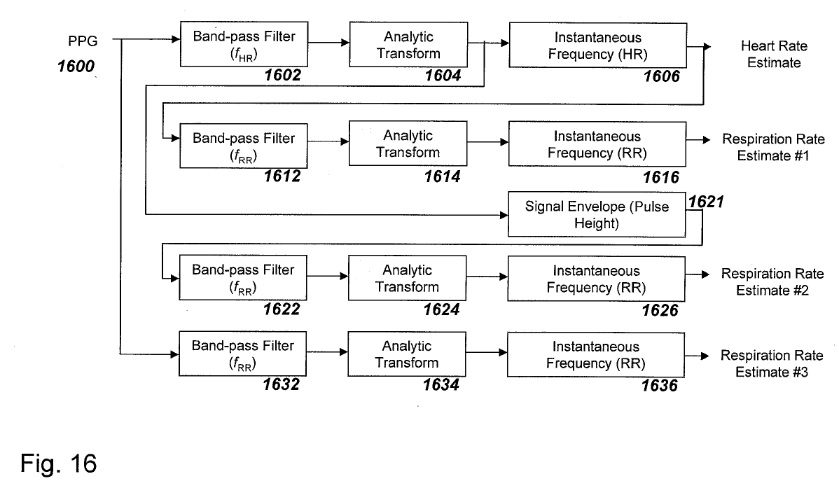

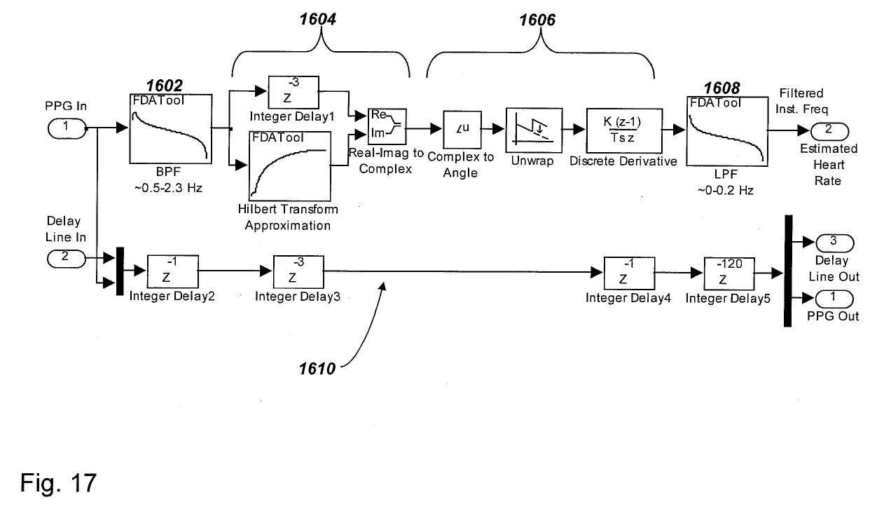

Referring to FIG. 16, a flow diagram shows various methods of estimating a heart rate and a respiration rate from data 1600 obtained from a PPG sensor. The procedure to obtain a heart rate estimate is described above in conjunction with FIG. 3: the data is band pass filtered (1602), an analytic transform is applied (1604), and the instantaneous frequency of the data is determined (1606). Referring to FIG. 17, an additional low-pass filter is applied (1608) to the data prior to output in order to smooth out any rapid variations. A delay line 1610 shows the lag of processing compared to real time. The delay corresponding to band pass filtering step 1602 is −1, the delay for the analytic transform is −3, the delay to apply the instantaneous frequency method is −1, and the delay for the final low pass filter is −120. For a sampling rate of 100 Hz, the determination of a heart rate estimate lags the input by 1.25 seconds.

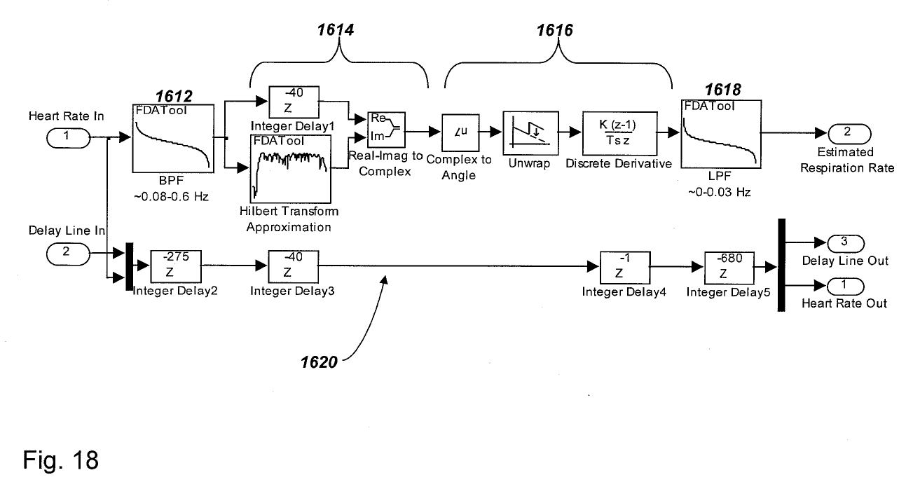

Referring again to FIG. 16, a first procedure to estimate a respiration rate based on the instantaneous frequency of the heart rate involves band pass filtering (1612) the instantaneous frequency data determined in step 1606. An analytic transform is applied (1614) and the instantaneous frequency is determined (1616). Referring to FIG. 18, an additional low pass filter is applied (1618) prior to output to smooth out any rapid variations in the signal. A delay line 1620 shows the lag of processing compared to real time. The delay corresponding to band pass filtering step 1612 is −275; that of the analytic transform 1614 is −40; that of the instantaneous frequency step 1616 is −1, and that of the low pass filter 1618 is −680. For a sample rate of 100 Hz, the determination of a respiration rate estimate based on the instantaneous frequency of the heart rate incurs a delay of 9.96 seconds.

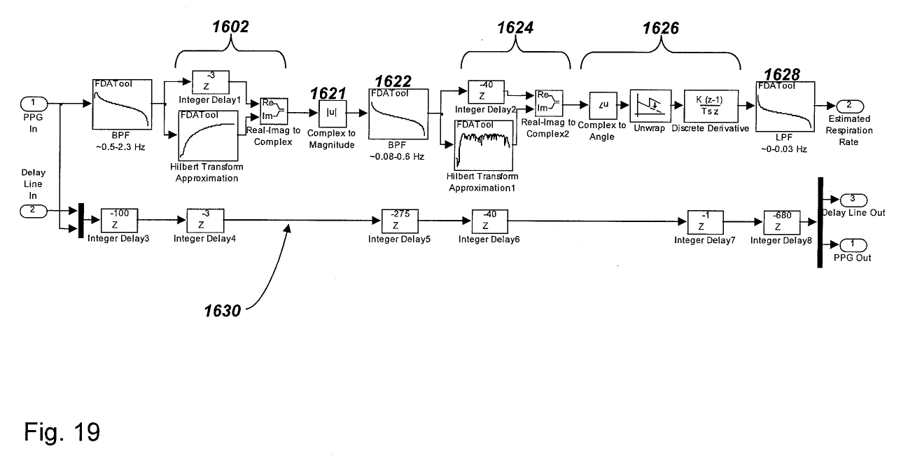

Referring again to FIG. 16, a second procedure to estimate a respiration rate based on an envelope 1621 (i.e., pulse height) of the heart rate involves a band pass filtering step (1622), an analytic transform (1624), and an application of the instantaneous frequency method (1626). Referring to FIG. 19, an additional low pass filter is applied (1628) to smooth any rapid variations in the output data. A delay line 1630 shows the lag of processing compared to real time. With the delays as shown, and for a sampling frequency of 100 Hz, the second procedure for the estimation of a respiration rate lags the input by 10.99 seconds.

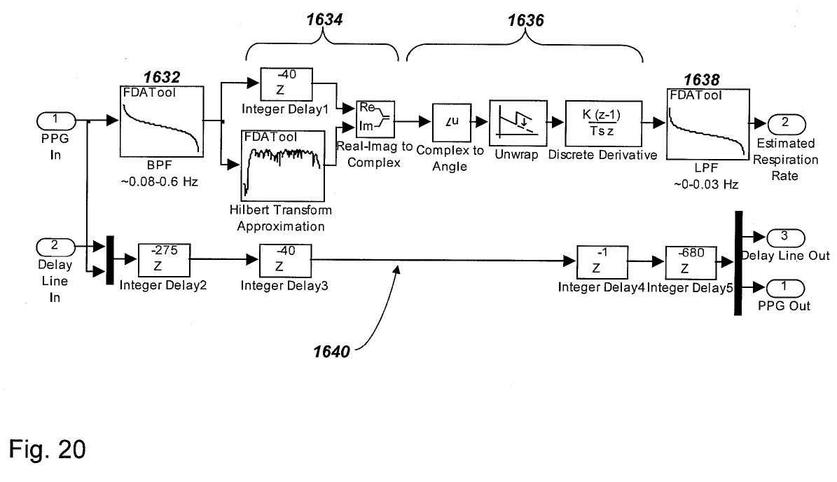

Referring again to FIG. 16, a third procedure to estimate a respiration rate based on an instantaneous frequency of a PPG signal 1600 involves a band pass filtering step (1632), an analytic transform (1634), and an application of the instantaneous frequency method (1636). Referring to FIG. 20, an additional low pass filter is applied (1638) to smooth any rapid variations in the output data. A delay line 1640 shows the lag of processing compared to real time. With the delays as shown, and for a sampling frequency of 100 Hz, the third procedure for the estimation of a respiration rate incurs a delay of 9.96 seconds.

Referring to FIG. 16, the three band pass filter steps 1612, 1622, and 1632 used to calculate the respiration rate use the same coefficients and the same calculation, with different inputs. Likewise, the three analytic transform steps 1614, 1624, and 1634 used to calculate the respiration rate use the same coefficients and the same calculation, with different inputs. The three instantaneous frequency steps 1616, 1626, and 1636 used to calculate the respiration rate also use the same coefficients and the same calculation, with different inputs.

Embodiments can be implemented using hardware (e.g., custom or semicustom circuitry, such as ASIC and FPGA) or software (e.g., instructions stored in a machine-readable device or medium for controlling a general purpose or custom processor such as a controller or signal processor) or a combination of hardware and software. As outlined above, in some embodiments, processing is performed solely in a small device that is attached to the body (e.g., clamped to the ear or finger, or applied as using an adhesive patch to the body), or can be distributed between a device attached in such a way and a second unit, such as a bedside or wearable unit. In some examples, the system is distributed over larger distances with elements of the system being couple, for example, over local or wide are data or telecommunication networks. Other embodiments are in the claims.

What is claimed is:

1. A system comprising:
an optical sensor configured to be positioned on an area of skin of a patient, the optical sensor including:
a light source for illuminating a capillary bed in the area of skin;
a photodetector configured to receive an optical signal from the capillary bed resulting from the illumination and to convert the optical signal into an electrical signal, the optical signal characterizing a fluctuation in a level of blood in the capillary bed; and
a signal processing module configured to process the electrical signal using a nonstationary frequency estimation method to obtain a processed signal related to at least one of a heart rate and a respiration rate of the patient.

2. The system of claim 1, further comprising an output for providing information determined from the processed signal.

3. The system of claim 1, wherein the nonstationary frequency estimation method comprises a Hilbert transform method.

4. The system of claim 1, wherein the nonstationary frequency estimation method comprises an instantaneous frequency estimation method.

5. The system of claim 1, wherein the processed signal comprises at least one of instantaneous heart rate, inter-beat interval, heart rate variability, high-low heart rate ratios, respiration rate, inter-breath interval, and respiration rate variability.

6. The system of claim 1, wherein the fluctuation in the level of blood in the capillary bed relates to a change in at least one of volume and pressure of the thoracic cavity.

7. The system of claim 1, wherein the fluctuation in the level of blood in the capillary bed relates to a change in at least one of volume and pressure of an organ in the thoracic cavity.

8. The system of claim 1, further comprising an auxiliary sensor configured to detect an ambient signal.

9. The system of claim 8, wherein the auxiliary sensor includes at least one of an accelerometer, a pressure sensor, an optical detector, a temperature sensor, and a piezoelectric device.

10. The system of claim 8, wherein the signal processing module is further configured to remove an effect of the ambient signal from the electrical signal.

11. The system of claim 1, wherein the optical signal is a reflectance of the capillary bed.

12. The system of claim 1, wherein the optical signal is a transmittance of the capillary bed.

13. A method comprising:
illuminating a capillary bed in an area of skin of a patient;
receiving an optical signal from the capillary bed resulting from the illumination;
converting the optical signal into an electrical signal, the optical signal characterizing a fluctuation in a level of blood in the capillary bed; and
processing the electrical signal using a nonstationary frequency estimation method to obtain a processed signal related to at least one of a heart rate and a respiration rate of the patient.

14. The method of claim 13, further comprising outputting information determined from the processed signal.

15. The method of claim 13, wherein processing the electrical signal using the nonstationary frequency estimation method comprises performing a Hilbert transform.

16. The method of claim 13, wherein processing the electrical signal using the nonstationary frequency estimation method comprises processing the electrical signal using an instantaneous frequency estimation method.

17. The method of claim 15, wherein processing the electrical signal using the instantaneous frequency method comprises:
band pass filtering the electrical signal;
determining an instantaneous frequency of the electrical signal; and
using the instantaneous frequency to obtain the processed signal.

18. The method of claim 13, further comprising:
processing the electrical signal using a model to obtain a blood pressure signal related to a blood pressure of the patient,
wherein the optical signal characterizes a capillary refill time in the capillary bed.

19. The method of claim 13, wherein processing the electrical signal includes processing the electrical signal in real time.

20. A method for monitoring a blood pressure of a patient, comprising:
illuminating a capillary bed in an area of skin of a patient;
receiving an optical signal from the capillary bed resulting from the illumination;
converting the optical signal into an electrical signal, the optical signal characterizing a fluctuation in a level of blood in the capillary bed of the patient; and
processing the electrical signal using a model characterizing a relationship of the fluctuation in the level of blood and the blood pressure of the patient to obtain a quantity related to the blood pressure of the patient.

21. The method of claim 20, further comprising outputting information determined based on the quantity related to the blood pressure of the patient.

22. The method of claim 20, wherein the optical signal characterizes a capillary refill time.

23. The method of claim 20, further comprising:
engaging a device to restrict circulation in the capillary bed of the patient; and
disengaging the device prior to receiving the optical signal from the capillary bed.

24. The method of claim 23, wherein the disengaging of the device occurs gradually.

25. The method of claim 23, wherein the device is an active clamping device.

26. The method of claim 20, wherein the quantity related to the blood pressure of the patient is a quantity related to the continuous blood pressure of the patient.

27. The method of claim 20, wherein applying the model comprises applying a model including circuit elements.

28. The method of claim 27, wherein applying the model further comprises applying a model including properties of the capillary bed.

29. The method of claim 20, further comprising calibrating the model on the basis of a blood pressure of the patient determined by using a blood pressure cuff.
